(19) United States
(12) Patent Application Publication
(10) Pub. No.: US 2011/0039794 A1
(43) Pub. Date: Feb. 17, 2011

(54) Long Acting Injectable Formulations

(76) Inventors: Carolina Nunes Costa Corgozinho, Joao Monlevade MG (BR); Karla de Melo Lima, Sao Paulo (BR); Jose Maciel Junior Rodriques, Sao Paulo (BR); Peter Andrew O'Neill, Seven Hills (AU)

Correspondence Address: Intervet/Schering-Plough Animal Health, Patent Dept. K-6-1, 1990, 2000 Galloping Hill Road, Kenilworth, NJ, 07033-0530, US

(21) Appl. No.: 12/739,300

(22) PCT Filed: Oct. 24, 2008
(86) PCT No.: PCT/EP08/64448

§ 371 Date: Apr. 22, 2010

Related U.S. Application Data
(60) Provisional application No. 61/095,015, filed on Sep. 8, 2008.

(30) Foreign Application Priority Data
Oct. 25, 2007 (BR) PI 0705822-5

(51) Int. Cl. A61K 31/365; A61P 33/14; A61P 33/10

(52) U.S. Cl. 514/28; 514/450

(57) ABSTRACT

Long acting injectable formulations of macrocyclic lactones comprising a biologically acceptable and biodegradable polyester polymer in a solvent system for use in the field of veterinary medicine, especially for use in combating ecto- and endoparasites in animals.

### FIELD OF THE INVENTION

This application relates to long acting injectable formulations of macrocyclic lactones comprising a biodegradable polyester polymer in a solvent or solvent system for use in the field of veterinary medicine, especially for use in combating ecto- and endoparasites in animals.

### BACKGROUND OF THE INVENTION

### Ecto- and Endo-Parasites of Animals

A number of pests and parasites can infest or infect livestock animals and also companion animals such as cats, dogs and horses. These pests and parasites are of great nuisance to both the animals and their owners.

Virtually all livestock and companion animals are affected by ectoparasites, i.e. arthropods which are injurious to, or spread or act as vectors of diseases in man and livestock and companion animals. Important arthropod parasites—ectoparasites (insect and acarid pests) are described below in more detail.

Biting insects include, e.g., migrating diperous larvae as Hypoderma sp. in cattle, Gastrophilus in horses, and Cuterebra sp. in rodents, as well as biting flies and mosquitoes spp of all types. For example, bloodsucking adult flies include, e.g., the horn fly or Haematobia irritans, the horse fly or Tabanus spp., the stable fly or Stomoxys calcitrans, the black fly or Simulium spp., the deer fly or Chrysops spp., the louse fly or Melophagus ovinus, the tsetse fly or Glossina spp. Parasitic fly maggots include, e.g., the bot fly (Oestrus ovis and Cuterebra spp.), the blow fly or Phaenicia spp., the screwworm or Cochliomyia hominivorax, the cattle grub or Hypoderma spp., and the fleeceworm. Mosquitoes, include, for example, Culex spp., Anopheles spp., and Aedes spp.

Mites include Mesostigmata spp. e.g., mesostigmatids such as the chicken mite, Dermanyssus gallinae; itch or scab mites such as Sarcoptidae spp. for example, Sarcoptes scabiei; mange mites such as Psoroptidae spp. including Chorioptes bovis and Psoroptes ovis; chiggers e.g., Trombiculidae spp.

Ticks include, e.g., soft-bodied ticks including Argasidae spp. for example Argas spp. and Ornithodoros spp.; hard-bodied ticks including Ixodidae spp., for example Ixodes ricinus, Rhipicephalus sanguineus, Haemaphysalis spp, Dermacentor reticulates, Dermacentor variabilis, Amblyomma americanum and Boophilus spp.

Lice include, e.g., sucking lice, e.g., Menopon spp. and Bovicola spp.; biting lice, e.g., Haematopinus spp., Linognathus spp. and Solenopotes spp.

Fleas include, e.g., Ctenocephalides spp., such as dog flea (Ctenocephalides canis) and cat flea (Ctenocephalides fells); Xenopsylla spp. such as oriental rat flea (Xenopsylla cheopis); and Pulex spp. such as human flea (Pulex irritans).

True bugs include, e.g., Cimicidae or e.g., the common bed bug (Cimex lectularius); Triatominae spp. including triatomid bugs also known as kissing bugs; for example Rhodnius prolixus and Triatoma spp.

Important endoparasites of animal hosts are parasitic worms known as helminths. Among the helminths, the group of worms described as nematodes causes widespread and often serious infection in various species of animals. The parasitic infections known as helminthiases lead to anemia, malnutrition, weakness, weight loss, severe damage to the walls of the intestinal tract and other tissues and organs, and, if left untreated, may result in death of the infected host. Helminthiasis is a prevalent and serious economic problem in domesticated animals such as swine, sheep, horses, cattle, goats, dogs, cats and poultry.

The most common genera of nematodes infecting the animals referred to above are Haemonchus, Trichostrongylus, Ostertagia, Nematodirus, Cooperia, Ascaris, Bunostomum, Oesophagostomum, Chabertia, Trichuris, Strongylus, Trichonema, Dictyocaulus, Capillaria, Heterakis, Toxocara, Ascaridis, Oxyuris, Ancylostoma, Uncinaria, Toxascaris and Parascaris. Certain of these, such as Nematodirus, Cooperia and Oesophagostomum, attack primarily the intestinal tract. Others, such as Haemonchus and Ostertagia, are more prevalent in the stomach. Others, such as Dictyocaulus, are found in the lungs. Still other parasites may be located in other tissues and organs of the body such as the heart and blood vessels, subcutaneous and lymphatic tissue, and the like.

### Macrocyclic Lactones

The macrocyclic lactones, i.e. avermectin and milbemycin series of compounds are potent endo- and ectoparasitic agents. The compounds which belong to this series are either natural products or are semi-synthetic derivatives thereof. The structure of these two series of compounds are closely related and they both share a complex 1,6-membered macrocyclic lactone ring; however, the milbemycins do not contain the disaccharide substituent in the 1,3-position of the lactone ring.

In a preferred embodiment of the invention, the macrocyclic lactones e.g. avermectins, milbemycins and derivatives thereof are selected from the group which includes but is not limited to, abamectin, doramectin, emamectin, eprinomectin, ivermectin, and selamectin (avermectin and derivatives thereof), milbemycin D, milbemycin oxime, lepimectin, and moxidectin (milbemycin and derivatives thereof) and mixtures thereof.

One particularly contemplated macrocyclic lactone parasiticide is ivermectin. Ivermectin is a semi-synthetic derivative of avermectin, and is generally produced as a mixture of at least 80% 22,23-dihydroavermectin B1ₐ and less than 20% 22,23-dihydroavermectin B1_{b}. Ivermectin is disclosed in U.S. Pat. No. 4,199,569. Ivermectin has been used as an antiparasitic agent to treat various parasitic diseases since the mid-1980's.

Other macrocyclic lactone parasiticides include, for example: Abamectin. This compound is, for example, identified as avermectin B1ₐ/B1_{b} in U.S. Pat. No. 4,310,519. Abamectin contains at least 80% of avermectin B1ₐ, and not more than 20% of avermectin B1_{b}. Doramectin. This compound is known as 25-cyclohexyl-avermectin B₁. Its structure and preparation are discussed in, for example, U.S. Pat. No. 5,089,480. Emamectin. This compound also is known as 4″-deoxy-4″-epi-methylaminoavermectin B₁. Its preparation is discussed in, for example, U.S. Pat. Nos. 5,288,710 and 5,399,717. Eprinomectin. This compound is known as 4″-epi-acetylamino-4″-deoxy-avermectin B₁. It was developed for use in all cattle classes and age groups. Selamectin. This compound also is known as 25-cyclohexyl-25-de(1-methyl propyl)-5-deoxy-22,23-dihydro-5-(hydroxyimino)-avermectin B1 monosaccharide. Milbemycin. This compound also is known as B41. It is isolated from the fermentation broth of a Milbemycin-producing strain of Streptomyces. The microorganism, fermentation conditions, and isolation procedures are discussed in, for example, U.S. Pat. Nos. 3,950,360 and 3,984,564. Moxidectin. This compound is discussed in, for example, U.S. Pat. No. 4,916,154. Lepimectin is a chemically modified milbemycin macrolide (6R,13R,25R)-5-O-demethyl-28-deoxy-6,28-epoxy-13-[(Z)-[(methoxyimino)phenylacetyl]oxy]-25-methylmilbemycin B mixture with (6R,13R,25R)-5-O-demethyl-28-deoxy-6,28-epoxy-25-ethyl-13-[(Z)-[(methoxyimino)phenylacetyl]oxy]milbemycin B.

While the individual macrocyclic lactones are well-known in the art, there have been difficulties in the art to provide for a viable, easy to use, long acting injectable formulation containing these endectoparasitic agents.

### Controlled-Release Technology

In the field of human and veterinary medicine many advantages are offered by controlled-, and especially prolonged release technology. First, controlled release of a pharmaceutical agent allows less frequent dosing and thus minimizes handling of animals. Further, controlled release treatment results in more efficient drug utilization. Further, less of the compound remains as a residue.

In the prior art many different concepts of prolonged release of injectable pharmaceutical compositions in animals have been described, e.g. use of low water soluble forms or complexes of active ingredients, use of liposome, microsphere and liposphere formulations, polymer formulations, oil based formulations, gel formulations etc.

Such concepts have been reviewed e.g. in Medlicott et al “Sustained release veterinary parenteral products”, Advanced Drug Delivery Reviews 56; (2004), p. 1345-1365, in Winzenburg et al” Biodegradable polymers and their potential use in parenteral veterinary drug delivery systems“, Advanced Drug Delivery Reviews 56; (2004), p. 1453-1466, in Matschke et al “Sustained-release injectables formed in situ and their potential use for veterinary products”, Journal of Controlled Release 85; (2002), p. 1-15 and in Packhaeuser et al “In situ forming parenteral drug delivery systems: an overview”, European Journal of Pharmaceutics and Biopharmaceutics 58; (2004), p. 445-455.

Despite these advantages, however, few prolonged release formulations for parenteral administration have been developed for commercial use in veterinary medicine.

Hence, there is still a need in the art for long acting formulations for prolonged release which are suitable for injection and which have long term shelf stability. In addition, an ideal injectable formulation would have a long acting effect that would have a season long effect during the breeding period for livestock mammals such as cattle, sheep, goats and pigs or minimize the number of injections when applying to companion mammals such as dogs, cats and horses.

### SUMMARY OF THE INVENTION

In view of the above-described state of the art, the objects of the invention are to provide a long acting injectable antiparasitic composition that combines the advantages of minimal repetitive administration, efficient drug utilization, and minimal handling.

Accordingly, a liquid long acting injectable formulation has been developed for the sustained release of macrocyclic lactones in animals, which includes the advantages of prolonged release of the macrocyclic lactones, easy manufacture and good stability.

An object of the invention is to provide a liquid long acting injectable formulation for combating ectoparasites and/or endoparasites. This object is achieved by formulations which comprise: (a) a therapeutically effective amount of a macrocyclic lactone; (b) a solvent or mixture of biologically acceptable solvents; and (c) a biologically acceptable and biodegradable polyester polymer.

Surprisingly, the liquid long acting injectable formulations of the invention solve the problems associated with previous injectable formulations by having long term stability in a liquid form thereby providing a convenient dosage form for achieving long acting effects in the control of endo- and ectoparasites of an animal.

These and other embodiments are disclosed or are apparent from and encompassed by the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 shows the mean adult tick counts after the application of a composition according to the invention compared to the closest prior art and control.

FIG. 2 shows the means by weight of tick counts in bovines after the administration of the present invention compared to the closest prior art and the control

FIG. 3 shows a comparison between the efficacy percentage of the parameters of number and weight of ticks after administration of a composition according to the invention compared to the closest prior art and the control

FIG. 4 shows a comparison between the mean number of Cooperia punctata recovered in necropsies of calves after the administration of a composition according to the invention compared to the closest prior art and control

FIG. 5 shows a comparison between the mean number of Trichuris discolor recovered in necropsies of calves after the administration of a composition according to the invention compared to the closest prior art and control

FIG. 6 shows the mean egg counts per gram of feces from calves after the administration of a composition according to the invention compared to the closest prior art and control

FIG. 7 shows the general average of helminthes in bovines after administration of a composition according to the invention compared to the closest prior art and control.

FIG. 8 shows the percentage tick, egg and egg hatch control in cattle after the application of a composition according to the invention.

FIG. 9 shows the blood plasma ivermectin concentration after administration of a composition according to the invention compared to the closest prior art.

### DETAILED DESCRIPTION

For the composition according to the current invention to present a long acting release profile there is a need to use a component capable of controlling the release of the macrocyclic lactone in a gradual manner, preferably at the injection site.

One embodiment of the invention is a liquid long acting injectable formulation for combating ectoparasites and/or endoparasites comprising a therapeutically effective amount of at least one macrocyclic lactone; a solvent or mixture of solvents; and at least one biologically acceptable and biodegradable polyester polymer.

A further embodiment of the liquid long acting injectable formulation is a formulation comprising a therapeutically effective amount of at least one macrocyclic lactone, a solvent that is selected from the group consisting of aromatic hydrocarbons, halocarbones; tetrahydrofuran, benzyl benzoate, benzyl alcohol, glycerol formal and mixtures thereof; and at least one biologically acceptable and biodegradable polyester polymer.

A further embodiment of the liquid long acting injectable formulation is a formulation where: (a) the macrocyclic lactone is selected from the group consisting of avermectins and milbemycins, derivatives thereof and mixtures thereof (b) the solvent is selected from aromatic hydrocarbons, halocarbons, tetrahydrofuran, caprolactone, benzyl benzoate, benzyl alcohol, glycerol formal and mixtures thereof (c) the biologically acceptable and biodegradable polyester polymer is selected from the group consisting of polyhydroxy acids, such as poly(lactide)s, poly(glycolide)s, poly(lactide-co-glycolide)s, poly(lactic acid)s, poly(glycolic acid)s, and poly(lactic acid-co-glycolic acid)s, polyanhydrides, polyorthoesters, polyetheresters, polyethylene glycol, polycaprolactone, polyesteramides, polyphosphazines, polycarbonates, polyamides, and copolymers and blends thereof.

A still further embodiment of the liquid long acting injectable formulation is a formulation where: (a) the macrocyclic lactone is selected from the group consisting of abamectin, doramectin, emamectin, eprinomectin, ivermectin, latidectin, lepimectin, and selamectin, milbemycin D, milbemycin oxime moxidectin and mixtures thereof (b) the solvent is selected from the group consisting of benzyl alcohol, and mixtures thereof with benzyl benzoate and/or, glycerol formal; (c) the biologically acceptable and biodegradable polyester polymer is selected from the group consisting of polylactides, poly-3-caprolactone or a copolyester of ε-caprolactone, polyglycolides and copolymers and blends thereof.

A yet further embodiment of the liquid long acting injectable formulation is a formulation where: (a) the macrocyclic lactone is selected from the group consisting of ivermectin, abamectin and moxidectin and mixtures thereof; (b) the solvent is selected from the group consisting of benzyl alcohol, and mixtures thereof with benzyl benzoate and/or, glycerol formal; (c) the biologically acceptable and biodegradable polyester polymer is poly-3-caprolactone or a copolyester of ε-caprolactone.

In another embodiment of the invention, the long acting injectable formulation of the invention has a therapeutic effect for a period of time selected from the group consisting of at least about three months to about one year, at least about three months to about six months and at least about three months to about five months

Another embodiment of the invention is directed to a process of making the liquid long acting injectable formulation of the invention which comprises: (i) dissolving the biologically acceptable polymer and biodegradable polyester polymer in a solvent to form a solution; (ii) adding a therapeutically effective amount of a macrocyclic lactone to the solution to form the formulation.

Alternatively the therapeutically effective amount of a macrocyclic lactone is first dissolved and then the biologically acceptable and biodegradable polyester polymer is added

Another embodiment of the invention is directed toward the method of combating ectoparasites and/or endoparasites in a mammal which comprises the parenteral administration of a therapeutically effective amount of the formulation of the invention to an animal in need thereof.

In one embodiment of the invention, the biologically acceptable and biodegradable polyester polymer can be any biologically acceptable and biodegradable polymer, such as recognized in documents cited herein. For instance, the biologically acceptable and biodegradable polyester polymer can have one or more or all of the following characteristics: be bioerodible by cellular action, biodegradable by action of non-living body fluid components, soften when exposed to heat but return to the original state when cooled or be capable of substantially dissolving or dispersing in a water-miscible or partially miscible carrier or solvent to form a solution or dispersion.

It is thought that upon contact with an aqueous fluids the polymers are capable of assisting in the formation of a film coated or encapsulated liquid.

The kinds of polyester polymers suitable for the present composition generally include any having the foregoing characteristics.

Biodegradable, as defined herein, means the polymer will degrade or erode in vivo to form smaller chemical species, wherein the degradation can result, for example, from enzymatic, chemical, and physical processes. The term “biologically acceptable” is used herein to refer to a polymer and any degradation products of the polymer that are non-toxic to a recipient and present no significant, deleterious or untoward effects on the recipient's body. Examples of suitable biologically acceptable and biodegradable polymers include polyhydroxy acids, such as poly(lactide)s, poly(glycolide)s, poly(lactide-co-glycolide)s, poly(lactic acid)s, poly(glycolic acid)s, and poly(lactic acid-co-glycolic acid)s, polyanhydrides, polyorthoesters, polyetheresters, polyethylene glycol, poly-ε-caprolactone, polyesteramides, polyphosphazines, polycarbonates, polyamides, and copolymers and blends thereof. Preferred materials are poly-ε-caprolactone (PCL), polyhydroxybutyrates and synthetic derivatives thereof, poly(lactide)s, poly(glycolide)s, and copolymers or blends thereof.

The biologically acceptable and biodegradable polyester polymer used in the present invention may be polyesters of lactic and glycolic acids (PLA and PLGA) or poly-ε-caprolactone.

In one embodiment the biologically acceptable and biodegradable polyester polymer is present in an amount of about 1 to about 25% w/v, especially about 5 to about 20% w/v. Even more advantageous are injectable formulations wherein the polymer is present in an amount of about 7.5% w/v.

The biologically acceptable and biodegradable polyester polymer may further contain blends consisting of the mixtures of the polymers above, that is blends of lactic acid and glycolic acid with PCL, or blends with different polyesters. The polymeric composition may further contain polymeric compositions with different molecular weights that may alter the viscosity and degradation time and consequently the kinetics of drug delivery at the injection site.

The solvent or solvent system comprises biologically acceptable solvents that are suitable for parenteral administration. In one embodiment of the invention the solvent is selected from a group of aromatic hydrocarbons, halocarbons, tetrahydrofuran, caprolactone, benzyl benzoate, benzyl alcohol, glycerol formal and mixtures thereof. An even more advantageous solvent is the group selected from benzyl alcohol, and mixtures thereof with benzyl benzoate and/or, glycerol formal.

An advantageous form for the above injectable formulations is where the solvent or mixtures of solvents is present in an amount of about 5 to about 95.0% w/v. An advantageous form for the above injectable formulations is where the benzyl alcohol is present in an amount of about 5 to about 95.0% w/v as sole solvent. Alternatively, the benzyl alcohol is present in an amount of about 5 to about 20.0% w/v, benzyl benzoate between about 1 to about 20.0% w/v and the reminder solvent qs is glycerol formal.

An advantageous form for the above injectable formulations is where the macrocyclic lactone is present in an amount of about 0.01w/v to about 50.0% w/v. Even more advantageous are injectable formulations wherein the macrocyclic lactone is present in an amount of about 1.0 w/v to about 20.0% w/v.

Especially advantageous are injectable formulations wherein the macrocyclic lactone is present in an amount of about 2.0 w/v to about 15.0% w/v. An especially advantageous amount for cattle products is where the macrocyclic lactone is present in an amount of about 4.0% w/v to about 10.0% w/v, even more advantageously, about 6.5% w/v.

Alternatively, the amount of macrocyclic lactone for the above injectable formulations can also be measured by the amount of macrocyclic lactone per bodyweight of the animal being treated. In this embodiment of the invention, the amount of macrocyclic lactone can range from about 0.01 to about 50 mg/kg. In an advantageous embodiment of the invention, the amount of bioactive agent ranges from about 0.05 mg/kg to about 10 mg/kg. In a particularly advantageous embodiment of the invention, the amount of bioactive agent ranges from about 0.1 mg/kg to about 5 mg/kg.

Since it is advantageous to have a ready to inject formulation as part of the invention, the amount of macrocyclic lactone can also be measured by the amount of macrocyclic lactone present in a unit of volume of injectable formulation. In this embodiment of the invention, the amount of bioactive agent can range from about 0.01 mg/mL to about 300 mg/mL. In an advantageous embodiment of the invention, the amount of bioactive agent ranges from about 0.1 mg/mL to about 150 mg/mL. In a particularly advantageous embodiment of the invention, the amount of bioactive agent ranges from about 5 mg/mL to about 100 mg/mL.

Optionally an antioxidant, such as e.g. BHA, (Butylated Hydroxyanisole) is present in the formulation. Other useful antioxidants include, for example, butylhydroxytoluene, ascorbic acid, sulphites, metabisulphites, or thiosulphates (e.g. sodium thiosulphate, sodium metabisulphite, potassium metabisulphite, etc.), propyl gallate, and/or tocopherol, or a mixture of not more than two of these agents.

The instant formulation is equally applicable to other compounds used for injection as long as such compounds are soluble or dispersed in the mixture of the solvent and biologically acceptable and biodegradable polyester polymer. Additional compounds that can be used in this formulation are other antiparasitic agents and antibiotics, therapeutic vitamin and mineral supplements, and other agents that are assisted in their therapeutic effect by having their effects extended over a prolonged period of time. Again, such compounds would be well known to the practitioner. Examples of antiparasitic agents include but are not limited to endoparasitics, such as benzimidazoles e.g. albendazole, fenbendazole or triclabendazole, or imidazothiazole anthelmintics such as levamisole, or pyrimidine anthelmintics such as pyrantel, or substituted phenols such as nitroxynil, or salicylanilides such as closantel or oxyclozanide or nodulosporic acid, depsipeptide or praziquantel. Examples of ectoparasitics are neonicotinoid pesticides such as imidacloprid, nitenpyram or dinotefuran, arylpyrazoles such as fipronil and Hoe 120739 or insect growth regulators such as azadirachtin, diofenolan, fenoxycarb, hydroprene, kinoprene, methoprene, pyriproxyfen, tetrahydroazadirachtin, chlorfluazuron, cyromazine, diflubenzuron, fluazuron, flucycloxuron, flufenoxuron, hexaflumuron, lufenuron, novaluron, tebufenozide, teflubenzuron, and triflumuron.

The composition conventionally further comprise physiologically acceptable formulation excipients known in the art e.g. as described in “Gennaro, Remington: The Science and Practice of Pharmacy” (20th Edition, 2000) incorporated by reference herein. All such components, carriers and excipients must be substantially pharmaceutically or veterinary pure and non-toxic in the amounts employed and must be compatible with the active ingredients.

The formulation according to the invention is useful in combating endo-and ectoparasite infestations of animals.

“Combating” means to alleviate or reduce parasite numbers in and/or on an animal, and/or to inhibit the development of parasite infestation in or on an animal, in whole or in part.

Control or “Efficacy” of a compound means that the parasite count is reduced, after a first administration, by an amount ranging from 5% to about 100%. The control of arthropods (e.g. insects, acarids) can be insecticidal, and/or acaricidal. The effect of the compounds of the invention can be e.g. ovicidal, larvicidal, nymphicidal and/or adulticidal or a combination thereof. The effect can manifest itself directly, i.e. killing the parasites either immediately or after some time has elapsed, for example when molting occurs, or by destroying their eggs, or indirectly, e.g. reducing the number of eggs laid and/or the hatching rate.

An “effective amount,” is the amount or quantity of a compound according to the invention that is required to alleviate or reduce parasite numbers in and/or on an animal, and/or to inhibit the development of parasite infestation in or on an animal, in whole or in part. This amount is readily determined by observation or detection of the parasite numbers both before and after contacting the sample of parasites including their stages with the compound, directly and/or indirectly, e.g., by contacting articles, surfaces, foliage, or animals with the compound.

For an in vivo administration of the compound according to the invention, an effective amount is synonymous with a “pharmaceutically effective amount” which is the dose or amount that treats or ameliorates symptoms and/or signs of parasite infection or infestation by the treated animal. This latter amount is also readily determined by one of ordinary skill in the art, e.g., by observing or detecting changes in clinical condition or behavior of treated animals, as well as by observing or detecting relative changes in parasite numbers after such treatment.

In another embodiment of the invention, the formulation of the invention is for combating endoparasites wherein the endoparasite is a helminth selected from the group consisting of Ancylostoina, Anecator, Ascaris, Capililaria, Cooperia, Dirofilaria, Dictyocaulus, Haemonchus, Oesophagostomum, Ostertagia, Toxocara, Strongyloides, Toxascaris, Trichinella, Trichuris, Trichostrongylus and mixtures thereof.

In a further embodiment of the formulation for combating ectoparasites, the ectoparasite is an insect or arachnid including those of the genera Ctenocephalides, Rhipicephalus, Dermacentor, Ixodes, Boophilus, Ambyloma, Hyaloma, Sarcoptes, Psoroptes, Otodectes, Chorioptes, Hypoderma, Damalinia, Linognathus, Hematopinus, Solenoptes.

Another embodiment of the invention for combating ectoparasites and/or endoparasites in a mammal is directed to having a therapeutic effect for a period of time selected from the group consisting of at least about three months to about one year, at least about three months to about six months and at least about three months to about five months.

The very high effectiveness of the method and of the composition according to the invention shows not only high instantaneous effectiveness but also an effectiveness of very long duration after the treatment of the animal. In one embodiment of the invention, the effectiveness of the long acting injectable formulations of the invention against pests is from about 1 day to about 90 days. In another advantageous embodiment of the invention, the effectiveness of the long acting injectable formulations of the invention against pests is from about 1 day to about 120 days. In the context of livestock animals such as cattle, pigs or sheep, about 120 days represents a season long treatment.

In a further advantageous embodiment of the invention, the effectiveness of the long acting injectable formulations of the invention against pests is from about 1 day to about 180 days. In a still further advantageous embodiment of the invention, the effectiveness of the long acting injectable formulations of the invention against pests is from about 1 day to about 365 days.

The formulation may be used to treat a range of animals, especially warm-blooded animals. Such warm-blooded animals include, for example, mammals. Mammals include, for example, humans. Other mammals include, for example, farm or livestock mammals (e.g., swine, bovines, sheep, goats, etc.), laboratory mammals (e.g., mice, rats, birds, etc.), companion mammals (e.g., dogs, cats, equines, etc.), fur-bearing animals (e.g., minks, foxes, chinchillas, rabbits, etc.), and wild and zoo mammals (e.g., buffalo, deer, etc.). In some embodiments, the compositions are used to treat canines (e.g., dogs, such as, for example, pure-bred and/or mongrel companion dogs, show dogs, working dogs, herding dogs, hunting dogs, guard dogs, police dogs, racing dogs, and/or laboratory dogs). In other embodiments, the compositions are used to treat felines (e.g., domestic cats). It is contemplated that the compositions also are suitable to treat non-mammals, such as birds (e.g., turkeys, chickens, geese, ducks, parrots, etc.). It is also contemplated that such compositions may be useful to treat cold-blooded animals as well, such as, for example, fish (e.g., salmon, trout, koi, etc.).

The invention will now be further described by way of the following non-limiting examples. It is not to be construed as a limitation of the invention.

### Examples

### Example 1

### Preparation of Compositions According to the Invention

A composition according to the invention was prepared using the following components:

Formulation 1:

| | | Concentration |
|---|---|---|
| Component | Function | (g/L) |
| Ivermectin | Active | 65.00 |
| Benzyl alcohol | Solvent | 180.00 |
| Benzyl benzoate | Solvent | 150.00 |
| BHA (Butylated | Antioxidant | 0.30 |
| Hydroxyanisole) | | |
| Polycaprolactone | Biodegradable | 75.00 |
| | Polymer | |
| Glycerol formal | Solvent | q.s. to 1 L |

The composition was obtained by dissolving the polycaprolactone polymer and ivermectin and BHA in the mixture of benzyl alcohol, benzyl benzoate and glycerolformal. Optionally the solution may be heated to a temperature between 30° C. to 70° C., which helps the dissolution of the components of the composition Optionally the composition is sterile filtered.

For the test 10 l of a solution with viscosity in the order of 200 cP was produced For the filtration step the formulation was heated to a temperature between 30° C. and 60° C. The sterilization filtration is optionally preceded by a coarser filtration step using filters with porosity above 1 μm. Therefore, there is no need to filter the formulation components in sterilizing filters before mixing of the components. Based on the filtration tests the following conclusions were reached:

Pre-filtration may occur by polypropylene elements and with pore size not greater than 1.0 micron.

The sterile filtration may be done by different polymeric filtering elements, presenting good chemical compatibility and

The ideal temperature for filtration is between 45° C. and 50° C.

The composition thus obtained was analyzed for microbial contamination according to the American Pharmacopoeia and was shown to be sterile. The lipopolysaccharide contamination test was determined by the L.A.L. method and has shown to be within the standards for injectable formulations.

The following alternative formulations were prepared

Formulation 2:

| | | Concentration |
|---|---|---|
| Component | Function | (g/L) |
| Moxidectin | Active | 100.00 |
| Benzyl alcohol | Solvent | 180.00 |
| Benzyl benzoate | Solvent | 150.00 |
| BHA | Antioxidant | 0.30 |
| (Butylated | | |
| Hydroxyanisole) | | |
| Polycaprolactone | Biodegradable Polymer | 75.00 |
| Glycerol formal | Solvent | 658.8 or q.s. to 1 L |

The stability of this formulation was tested after 2 months storage at 2-4° C., 30° C. 65% RH, 40° C. 75% RH and 45° C. 75% RH and it was found to be stable.

Formulation 3:

| | | Concentration |
|---|---|---|
| Component | Function | (g/L) |
| Ivermectin | Active | 65.00 |
| BHA | Antioxidant | 0.30 |
| (Butylated | | |
| Hydroxyanisole) | | |
| Polycaprolactone | Biodegradable Polymer | 75.00 |
| Benzyl Alcohol | Solvent | 910.3 or q.s. to 1 L |

The composition was obtained by dissolving the polycaprolactone polymer ivermectin and BHA in the benzyl alcohol. Optionally the solution may be heated to a temperature between 30° C. to 70° C., which helps the dissolution of the components of the composition Optionally the composition is sterile filtered.

The stability of this formulation was tested after 1 month storage at 2-4° C., 30° C. 65% RH, 40° C. 75% RH and 45° C. 75% RH and it was found to be stable.

Formulation 4:

| | | Concentration |
|---|---|---|
| Component | Function | (g/L) |
| Ivermectin | Active | 65.00 |
| Abamectin | Active | 35.00 |
| BHA | Antioxidant | 0.30 |
| (Butylated | | |
| Hydroxyanisole) | | |
| Polycaprolactone | Biodegradable Polymer | 75.00 |
| Benzyl Alcohol | Solvent | 873.03 or q.s. to 1 L |

The stability of this formulation was tested after 1 month storage at 2-4° C., 30° C. 65% RH, 40° C. 75% RH and 45° C. 75% RH and it was found to be stable.

Formulation 5:

| | | | Concentration |
|---|---|---|---|
| Component | CAS Number | Function | (g/L) |
| vermectin | 70288-86-7 | Active | 100.00 |
| BHA | 25013-16-5 | Antioxidant | 0.30 |
| (Butylated | | | |
| Hydroxyanisole) | | | |
| Polycaprolactone | 25248-42-4 | Biodegradable | 75.00 |
| | | Polymer | |
| Benzyl Alcohol | 100-51-6 | Solvent | 876.8 or q.s. to 1 L |

The stability of this formulation was tested after 1 month storage at 2-4° C., 30° C. 65% RH, 40° C. 75% RH and 45° C. 75% RH and it was found to be stable.

| | | |
|---|---|---|
| | | Concentration |
| Component | Function | (g/L) |

Contemplated additional formulations: Formulation 6:

| | | |
|---|---|---|
| Ivermectin | Active | 65.00 |
| Benzyl alcohol | Solvent | 150.00 |
| Benzyl benzoate | Solvent | 150.00 |
| Polycaprolactone | Biodegradable | 75.00 |
| | Polymer | |
| Glycerol formal | Solvent | q.s. to 1 L |

Formulation 7:

| | | |
|---|---|---|
| Ivermectin | Active | 65.00 |
| Glycerol formal | Solvent | 180.00 |
| Benzyl benzoate | Solvent | 150.00 |
| Polycaprolactone | Biodegradable | 75.00 |
| | Polymer | |
| Benzyl alcohol | Solvent | q.s. to 1 L |

### Example 2

### Formulation Stability Study

Compositions of Example 1-Formulation 1—were exposed to different temperatures and humidity conditions and analyzed by HPLC methods according to American Pharmacopoeia USP 28. The samples were stored in a climatic chamber and kept for a period of 36 months.

Tables 1 and 2 depict the results obtained for accelerated stability studies (50° C. and 90% RH) and for long term stability studies (30° C. and 65% RH) of Formulation 1. Table 3 shows the results of the multidose stability study with this composition Formulation 1. The composition has been shown to be stable, no content reduction beyond 5% was observed, even under severe temperature and humidity conditions. In addition the product remained sterile during the test period.

TABLE 1
Accelerated Stability Study - Ivermectin
content (% LC = Label Claim)
50° C./90% RH

| | Start Time | 30 days | 60 days | 90 days |
|---|---|---|---|---|
| 500 ml vial | 6.57% | 6.82% | 6.93% | 6.64% |
| | (101.1% LC) | (104.9% LC) | (106.6% LC) | (102.2% LC) |
| 1000 ml vial | 6.64% | 6.75% | 6.86% | 6.89% |
| | (102.1% LC) | (103.9% LC) | (105.5% LC) | (104.7% LC) |

TABLE 2
Long-Term Stability Study - Ivermectin content (% LC = Label Claim)
30° C./65% RH

| | Start Time | 90 days | 6 months | 9 months | 12 months |
|---|---|---|---|---|---|
| 500 ml vial | 6.57% | 6.84% | 6.869% | 6.60% | 7.21% |
| | (101.1% LC) | (105.2% LC) | (105.5% LC) | (101.5% LC) | (110.9% LC) |
| 1000 ml vial | 6.64% | 6.44% | 7.24% | 6.41% | 6.93% |
| | (102.1% LC) | (99.0% LC) | (111.4% LC) | (98.6% LC) | (106.6% LC) |

TABLE 3
Multidose Stability Study - Ivermectin content
(% LC = Label Claim)
30° C./65% RH

| | Start Time | 3 months | 6 months | 9 months |
|---|---|---|---|---|
| 500 ml vial | 6.57% | 6.91% | 7.20% | 6.57% |
| | (101.1% LC) | (106.3% LC) | (110.7% LC) | (100.1% LC) |
| 1000 ml vial | 6.64% | 6.43% | 6.40% | 6.45% |
| | (102.1% LC) | (98.9% LC) | (98.4% LC) | (99.2% LC) |

### Example 3

### Efficacy Study Against Ecto- and Endoparasites

### Example 3.1

### Tick Efficacy

The Efficacy of the composition according to Example 1-Formulation 1—comprising 6.5% ivermectin (Formulation A) was compared with a prior art formulation comprising 3.15% ivermectin (Formulation B—Ivomec Gold-closest prior art) and an ivermectin free control

The compositions were administered subcutaneously to 13 bovines of Aberdeen breed (Angus and Red) aged between 10 and 14 months, that were infested with Boophilus microplus larvae, aged 10 and 30 days after eclosion. These larvae are sensitive to phosphate, pyrethroid and amidinic compounds.

The animals were infested 3 times a week during 2.5 consecutive weeks, totaling 10 infestations before the start of the treatment.

The compositions were administered at a dosage of 1 ml/100kg for formulation A and 1 ml/50 kg for formulation B.

After treatment the animals were weekly infested with larvae until the recurrence of adult ticks on day 67 post treatment. The collection of ticks started on Day 19. For calculating the efficacy in relation to the number of ticks, the following formula was used:

100−treated ticks/control×100

For calculating the efficacy in relation to the weight of ticks, the following formula was used:

100−mass of treated ticks/control×100

As shown in FIG. 1 the average number of ticks between Day 29 and 66 post treatment in the treated group was around zero. Formulation A corresponds to S 6.5% in FIG. 1

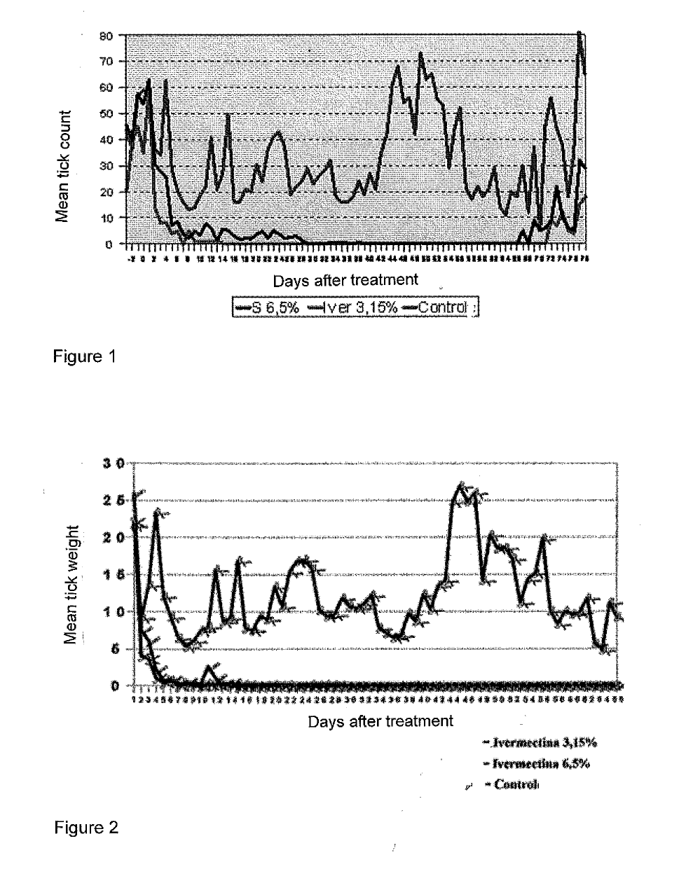

As indicated in FIG. 2 a reduction of mean weight of the ticks was observed in the treated group from Day 3 on. Formulation A corresponds to Ivermectin 6.5% in FIG. 2

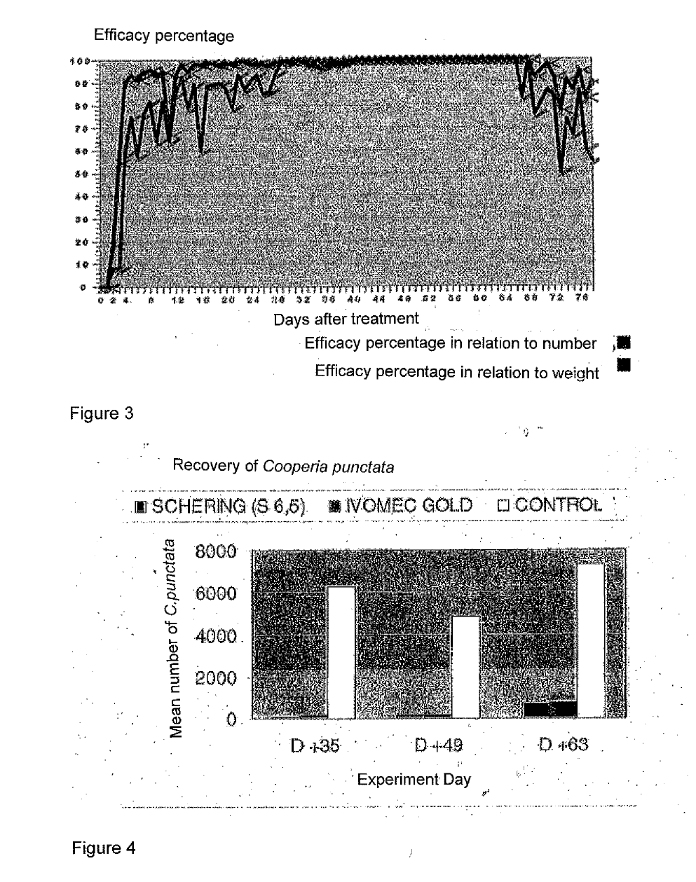

The treatment group showed an average tick control efficacy of 95.52% between days 4 and 28 post treatment and 99.15% between days 29 and 68 post treatment against Boophilus microplus ticks in a stable test (FIG. 3)

### Example 3.2

### Nematode Efficacy

The Efficacy of the composition according to Example 1-Formulation 1 comprising 6.5% ivermectin (Formulation A) was compared with a prior art formulation comprising 3.15% ivermectin—Ivomec Gold, Merial (Formulation B—closest prior art) and an ivermectin free control.

The compositions were administered subcutaneously to 72 hybrid Dutch×Zebu calves between 7 and 10 months old, that were infested with approximately 2000 infecting larvae of gastrointestinal and pulmonary nematodes, originating from a mixed culture.

The compositions were administered at a dosage of 1 ml/100kg for formulation A and 1 ml/50 kg for formulation B.

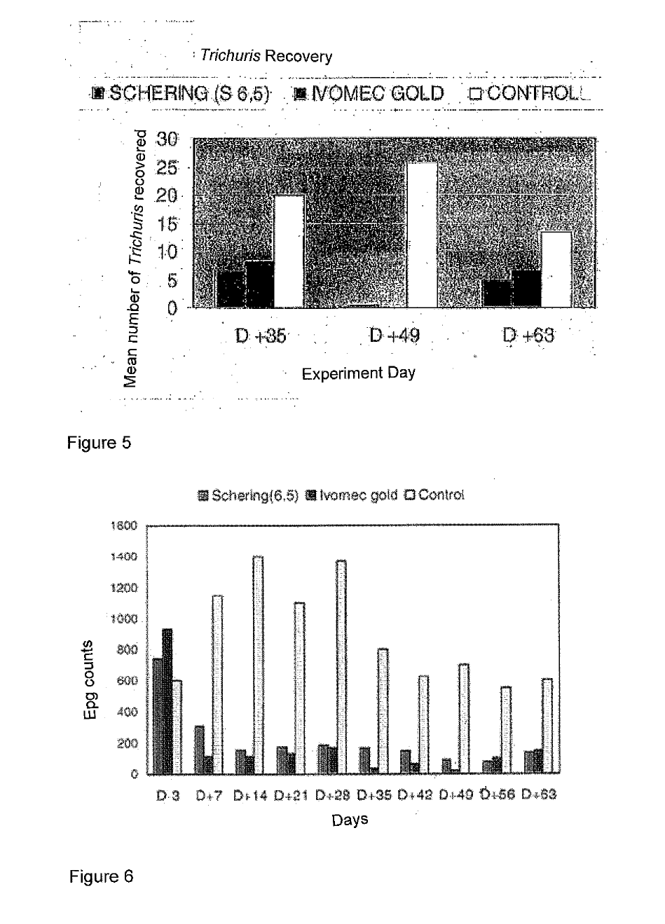

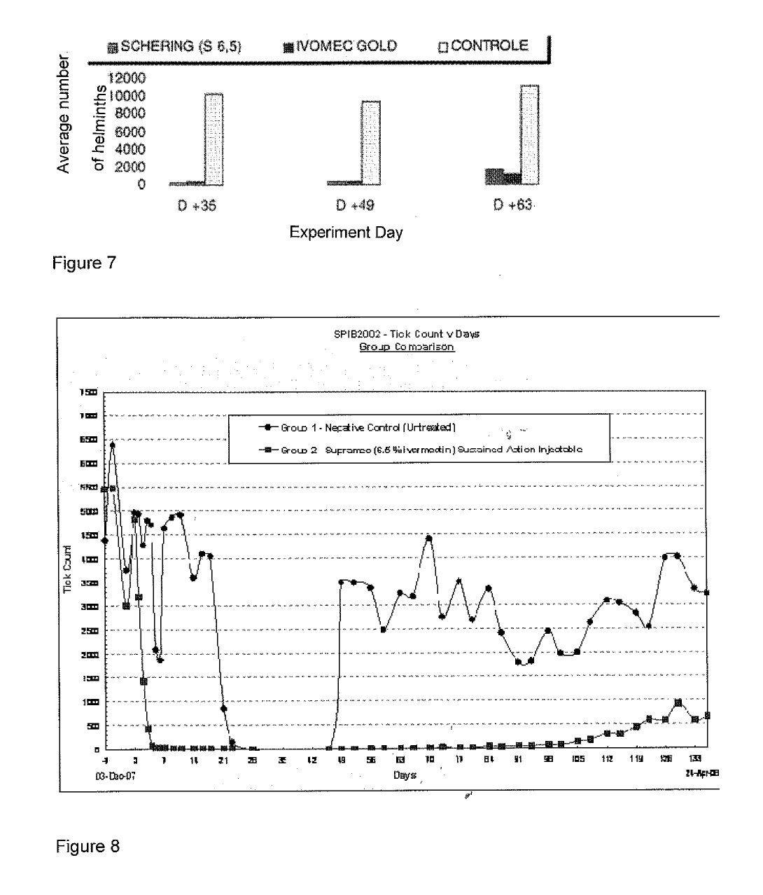

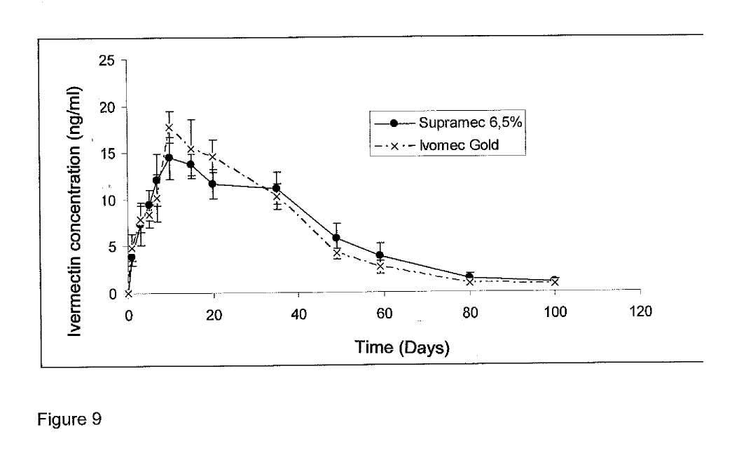

The results of the evaluation after necropsy of the animals is illustrated in the Table 4 below and FIGS. 4 to 7. Formulation A corresponds to SCHERING (S 6.5) in FIGS. 4 to 7,

### Example 3.3

### Efficacy, Period of Persistent Activity for Control of Artificially Acquired Cattle Tick Burdens

A single pen trial was conducted in cattle to determine the persistent efficacy of the Formulation A against an artificially acquired burden of cattle tick (Boophilus microplus).

Sixteen Hereford male castrate cattle were divided into two groups, each of eight animals and from Day −28 to Day −3 each animal was artificially infested with 5000 non-resistant field strain (NRFS) Boophilus microplus larval ticks on twelve separate occasions. This ensured that all parasitic stages, i.e. larval, nymph and adult stages, of B. microplus were present on the animal at the time of treatment.

Cattle in Group 2 were treated with Formulation A as test formulation on Day 0.

Cattle in group 1 remained as untreated negative controls. All treatments were administered subcutaneously according to individual body weight on the day of treatment at a dose rate of 0.65mg/kg ivermectin (0.1 ml/10 kg).

From Day 28 to Day 115 following treatment, cattle were artificially infested twice weekly with 5000 NRFS Boophilus microplus to assess the persistent efficacy of the test formulation.

From Day −7 to Day 28 and from Day 46 to day 136 post treatment, all engorged adult female ticks dropping from cattle were collected and then counted and weighted. A sample of 10 engorged female ticks was incubated and the viability of egg hatches assessed visually. Efficacy was determined by standard ADEQ analysis method.

The results are illustrated in FIG. 8. Results of the study indicated that test formulation A when administered to cattle as a s.c. injection, at a dose rate of approximately 1 ml/100 kg was highly effective against all stages of B. microplus particularly the larval and nymph stages; and provided protection against re-infesting larvae for a period of 66 days. Effective daily group control (at least 98% efficacy) was from Day 4 to Day 87 for tick control and Day 3 to Day 115 for both egg control and egg control at hatch.

TABLE 4
Number of gastrointestinal helminthes recovered from necropsy of 6 animals per group (Group A, B and Control) on Day 35

| | C. punctata | | C. pectinata | | H. contortus | | T. axei | | O. radiatum | | T. discolor | | B. phlebotomum | | |
|---|---|---|---|---|---|---|---|---|---|---|---|---|---|---|---|
| Animal | Im | Ad | Im | Ad | Im | Ad | Im | Ad | Im | Ad | Im | Ad | Im | Ad | Total |

Group A

| | | | | | | | | | | | | | | | |
|---|---|---|---|---|---|---|---|---|---|---|---|---|---|---|---|
| 200- | 0 | 20 | 0 | 0 | 0 | 30 | 0 | 0 | 0 | 10 | 0 | 20 | 0 | 0 | 80 |
| 005Az | | | | | | | | | | | | | | | |
| 187- | 0 | 0 | 0 | 0 | 1 | 105 | 0 | 0 | 0 | 0 | 0 | 0 | 0 | 0 | 106 |
| 016Az | | | | | | | | | | | | | | | |
| 122- | 20 | 80 | 0 | 0 | 4 | 186 | 0 | 0 | 0 | 20 | 0 | 0 | 0 | 0 | 310 |
| 010Az | | | | | | | | | | | | | | | |
| 57-87Az | 0 | 460 | 0 | 0 | 0 | 165 | 0 | 0 | 0 | 10 | 0 | 0 | 0 | 0 | 635 |
| 198- | 0 | 10 | 0 | 0 | 0 | 120 | 0 | 0 | 0 | 0 | 0 | 20 | 0 | 0 | 150 |
| 007Az | | | | | | | | | | | | | | | |
| 41-011Az | 0 | 160 | 0 | 50 | 14 | 53 | 0 | 0 | 0 | 0 | 0 | 0 | 0 | 0 | 277 |
| Total | 20 | 730 | 0 | 50 | 19 | 659 | 0 | 0 | 0 | 40 | 0 | 40 | 0 | 0 | 1588 |

Group B

| | | | | | | | | | | | | | | | |
|---|---|---|---|---|---|---|---|---|---|---|---|---|---|---|---|
| 080- | 0 | 30 | 0 | 0 | 0 | 113 | 0 | 0 | 0 | 30 | 0 | 0 | 0 | 0 | 173 |
| 052ver | | | | | | | | | | | | | | | |
| 054- | 20 | 260 | 0 | 0 | 0 | 108 | 0 | 0 | 10 | 60 | 0 | 0 | 0 | 0 | 458 |
| 117ver | | | | | | | | | | | | | | | |
| 179- | 0 | 0 | 0 | 0 | 0 | 60 | 0 | 0 | 0 | 0 | 0 | 0 | 0 | 0 | 60 |
| 198ver | | | | | | | | | | | | | | | |
| 035- | 5 | 246 | 1 | 50 | 0 | 113 | 0 | 0 | 0 | 10 | 20 | 20 | 0 | 0 | 465 |
| 122ver | | | | | | | | | | | | | | | |
| 125- | 8 | 127 | 0 | 0 | 10 | 82 | 0 | 0 | 0 | 0 | 0 | 10 | 0 | 0 | 273 |
| 122ver | | | | | | | | | | | | | | | |
| 033- | 0 | 250 | 0 | 30 | 14 | 210 | 0 | 0 | 0 | 20 | 0 | 0 | 0 | 0 | 524 |
| 199ver | | | | | | | | | | | | | | | |
| Total | 33 | 913 | 1 | 80 | 24 | 686 | 0 | 0 | 10 | 120 | 20 | 30 | 0 | 0 | 1917 |

Control

| | | | | | | | | | | | | | | | |
|---|---|---|---|---|---|---|---|---|---|---|---|---|---|---|---|
| 177- | 143 | 7701 | 0 | 0 | 170 | 5860 | 0 | 60 | 10 | 141 | 0 | 30 | 0 | 0 | 14115 |
| 145Am | | | | | | | | | | | | | | | |
| 036- | 150 | 4654 | 20 | 410 | 20 | 2547 | 0 | 60 | 0 | 100 | 0 | 23 | 0 | 0 | 7984 |
| 135Am | | | | | | | | | | | | | | | |
| 174- | 650 | 7260 | 80 | 1140 | 30 | 2070 | 0 | 180 | 30 | 142 | 0 | 0 | 0 | 0 | 11602 |
| 192Am | | | | | | | | | | | | | | | |
| 114- | 300 | 10734 | 0 | 0 | 1000 | 1840 | 13 | 323 | 400 | 620 | 0 | 57 | 10 | 20 | 15297 |
| 139Am | | | | | | | | | | | | | | | |
| 154- | 230 | 2350 | 10 | 180 | 30 | 420 | 0 | 45 | 0 | 0 | 0 | 10 | 0 | 0 | 3275 |
| 052Am | | | | | | | | | | | | | | | |
| 175- | 530 | 3000 | 53 | 810 | 1050 | 2580 | 0 | 130 | 101 | 1000 | 0 | 0 | 1 | 61 | 9316 |
| 199Am | | | | | | | | | | | | | | | |
| Total | 2003 | 35699 | 163 | 2540 | 2300 | 15317 | 13 | 798 | 541 | 2003 | 0 | 120 | 11 | 81 | 61589 |

Im = Parasites in immature stages,
Ad = Parasites in adult stage

1. A liquid long acting injectable formulation for combating ectoparasites and endoparasites in an animal comprising a therapeutically effective amount of at least one macrocyclic lactone, a solvent that is selected from the group consisting of aromatic hydrocarbons, halocarbons;
tetrahydrofuran, benzyl alcohol, benzyl benzoate, glycerol formal and mixtures thereof; and at least one biologically acceptable and biodegradable polyester polymer.

2. The formulation of claim 1, wherein: the biologically acceptable and biodegradable polyester polymer is selected from the group consisting of polyhydroxy acids, such as poly(lactide)s, poly(glycolide)s, poly(lactide-co-glycolide)s, poly(lactic acid)s, poly(glycolic acid)s, and poly(lactic acid-co-glycolic acid)s, polyanhydrides, polyorthoesters, polyetheresters, polyethylene glycol, polycaprolactone, polyesteramides, polyphosphazines, polycarbonates, polyamides, and copolymers and blends thereof.

3. The formulation of claim 2, wherein the biologically acceptable and biodegradable polyester polymer is selected from the group consisting of polylactides, polycaprolactones, polyglycolides and copolymers thereof.

4. The formulation of claim 1, wherein the biologically acceptable and biodegradable polyester polymer is ε-polycaprolactone.

5. The formulation of claim 1, wherein the solvent is selected from benzyl alcohol, and mixtures thereof with benzyl benzoate and/or, glycerol formal.

6. The formulation of claim 5, wherein the solvent is benzyl alcohol.

7. The formulation of claim 1, wherein the macrocyclic lactone is selected from the group consisting of abamectin, doramectin, emamectin, eprinomectin, ivermectin, lepimectin, and selamectin, milbemectin, milbemycin D, milbemycin oxime, moxidectin and mixtures thereof.

8. The formulation of claim 7, wherein the macrocyclic lactone is selected from the group consisting of ivermectin, abamectin, moxidectin and mixtures thereof.

9. The formulation of claim 8, wherein the macrocyclic lactone is ivermectin.

10. The formulation of claim 1 consisting essentially of ivermectin, abamectin or mixtures thereof, benzyl alcohol, and ε-polycaprolactone.

11. The formulation of claim 1 consisting essentially of ivermectin, abamectin or mixtures thereof, benzyl alcohol, benzyl benzoate glycerol formal and ε-polycaprolactone.

12-13. (canceled)

14. A method of combating ectoparasites and/or endoparasites in a mammal which comprises of parenteral administration of a therapeutically effective amount of the liquid long acting injectable formulation of claim 1 to an animal in need thereof.

15. The method of claim 14, wherein the combating of ectoparasites and/or endoparasites has a therapeutic effect for a period of time of at least about three months to about one year.

16. The method of claim 15, wherein the therapeutic effective period of time is at least about three months to about six months.

17. The method of claim 15, wherein the therapeutic effective period of time is at least about three months to about five months.
